(19) United States
(12) Patent Application Publication
(10) Pub. No.: US 2026/0355481 A1
(43) Pub. Date: Nov. 12, 2026

(54) HANDHELD DEVICE FOR APPLICATION OF NANOFIBER MATRIX FOR WOUND CARE

(71) Applicant: Matregenix, Inc., Mission Viejo, CA (US)

(72) Inventor: Sherif Soliman, Irvine, CA (US)

(73) Assignee: Matregenix, Inc., Mission Viejo, CA (US)

(21) Appl. No.: 19/746,494

(22) Filed: Jul. 17, 2026

Related U.S. Application Data
(63) Continuation-in-part of application No. 19/540,757, filed on Feb. 15, 2026.
Application No. 19/540,757 is a continuation of application No. PCT/US2024/042587, filed on Aug. 15, 2024.
(60) Provisional application No. 63/519,839, filed on Aug. 15, 2023.
(60) Provisional application No. 63/624,280, filed on Jan. 23, 2024.

(51) Int. Cl. A61M 35/00; D01D 1/02; D01D 5/00

(52) U.S. Cl. CPC A61M 35/003; D01D 1/02; D01D 5/0046; A61M 2205/3327; A61M 2205/50; A61M 2205/8206; A61M 2210/04; D10B 2321/06; D10B 2509/022

(57) ABSTRACT

The present disclosure describes a handheld device for application of a nanofiber matrix for wound care. In some implementations, the device includes an electrospinning apparatus to generate a nanofiber matrix for wound dressing and tissue regeneration. In some implementations, the device may include a ring that is positioned over on in front of a needle or other nozzle used for electrospinning. The ring may be configured to narrow and focus the electrospinning jet, facilitating more accurate targeting of a patient's wound. In some implementations, the device may include a microcontroller that may be used to make real-time adjustments to electrospinning parameters during the electrospinning process. In some implementations, the device may include an electro-centrifugal spinning apparatus. The electro-centrifugal spinning apparatus may include a spinneret that is operationally coupled to multiple nozzles for electro-centrifugal spinning. The electro-centrifugal spinning apparatus may further include a rotation arm that causes rotation of the nozzles.

### CROSS-REFERENCE TO RELATED APPLICATIONS

This application is a continuation-in-part of U.S. patent application Ser. No. 19/540,757, filed on Feb. 15, 2026, which is a continuation of PCT Patent Application No. PCT/US2024/042587, filed on Aug. 15, 2024, which claims the benefit of U.S. Provisional Patent Application Ser. Nos. 63/519,839, filed on Aug. 15, 2023, and 63/624,280, filed on Jan. 23, 2024, the disclosures of which are hereby incorporated in their entireties herein by reference.

### BACKGROUND

### Field of the Invention

The present disclosure relates to handheld devices for use in wound care.

### Description of the Related Art

Severe burn wounds are responsible for about 40,000 injuries requiring hospitalization each year in the United States. See Norbury, W., et al. “Infection in Burns,” Surg. Infect. (Larchmt), 2016, 17(2), 250-5, doi: 10.1089/sur.2013.134. It has been estimated that 10-12% of injuries in the military during conflicts are burn injuries. See Stokes, M. A. R., et al. “Burns in the Third World: An Unmet Need,” Ann. Burns Fire Disasters, 2017, 30(4), 243-46. As the burn care industry continues to develop new and better treatments, burn patients are becoming more aware of enhanced and diversified treatment alternatives that minimize the pain and inconvenience of changing bandages or enduring excruciating surgical procedures for skin grafting. Moreover, children under 16 account for approximately 26% of all admissions to burn center hospitals in the United States. See Am. Burn Ass'n, “Scald Statistics and Data Resources,” 2018, 1, available at: https://ameriburn.org/wp-content/uploads/2018/12/nbaw2019_statsdataresources_120618-1.pdf.

Given the pain associated with wound treatment and the importance of a patient remaining still during treatment, a contactless and rapid therapeutic technique is highly desirable. In addition, non-fatal burn wounds contribute to extended hospital stays, disfigurement, and disability. According to the World Health Organization, burns are also a leading cause of loss in disability-adjusted life-years (DALYs) among lower to middle income countries. See Rybarczyk, M. M., et al. “A Systematic Review of Burn Injuries in Low- and Middle-Income Countries: Epidemiology in the WHO-Defined African Region,” Afr. J. Emerg. Med. 2017, 7(1), 30-37, doi: 10.1016/j.afjem.2017.01.006. In lower to middle-income communities across America, constrained living circumstances and lack of access to rapid emergency responses or proper hospital care creates a risk of delayed treatment. Delayed access to burn wound treatment and emergency surgery increases the likelihood of disfigurement, sepsis, delayed healing, and increased mortality. Therefore, there is a significant need for a portable and easy-to-use wound dressing device that is readily accessible and can be used inside and outside of a hospital, in an ambulance, or at the site of the burn-causing accident.

### SUMMARY

The present disclosure describes a handheld device for application of a nanofiber matrix for wound care. In some implementations, the device includes an electrospinning apparatus to generate a nanofiber matrix for wound dressing and tissue regeneration.

In some implementations, the device may include a ring that is positioned over on in front of a needle or other nozzle used for electrospinning. The ring may be configured to narrow and focus the electrospinning jet, facilitating more accurate targeting of a patient's wound.

In some implementations, the device may include a microcontroller that may be used to make real-time adjustments to electrospinning parameters during the electrospinning process.

In some implementations, the device may include an electro-centrifugal spinning apparatus. The electro-centrifugal spinning apparatus may include a spinneret that is operationally coupled to multiple nozzles for electro-centrifugal spinning. The electro-centrifugal spinning apparatus may further include a rotation arm that causes rotation of the nozzles.

### BRIEF DESCRIPTION OF THE DRAWINGS

FIG. 1 shows an embodiment of the disclosed device as an application gun that includes an electrospinning apparatus.

FIGS. 2A and 2B show embodiments of a ring positioned near the front of a nozzle of an embodiment of the disclosed device from which an electrospinning jet emerges when the device is in use.

FIG. 3 shows an embodiment of the disclosed electro-centrifugal spinning apparatus.

FIG. 4 shows a close-up sectional view of the embodiment shown in FIG. 3.

FIG. 5 shows an embodiment of the disclosed device as an applicator that includes an electrospinning apparatus.

FIG. 6 shows an example of a set of parameters that are communicatively coupled to a microcontroller, where operating parameters are adjusted algorithmically

FIG. 7A shows a schematic representation of the configuration of a convergent-divergent nozzle.

FIG. 7B shows an example of a convergent-divergent nozzle.

### DETAILED DESCRIPTION

The present disclosure describes a handheld device for application of a nanofiber matrix for wound care. In some implementations, the device includes an electrospinning apparatus to generate a nanofiber matrix for wound dressing and tissue regeneration. In some alternate implementations, the device uses electrospinning, centrifugal spinning, or electro-centrifugal spinning techniques to generate a nanofiber matrix for wound dressing and tissue regeneration.

The disclosed device generates a nanofiber matrix that is placed or adhered directly onto target tissues. This facilitates cellular adhesion, proliferation, and tissue regeneration. The handheld design enhances practicality for point-of-care applications, empowering healthcare professionals to administer precise and controlled treatments for tissue engineering and wound healing. The device may be used in regenerative medicine, wound care, and tissue engineering applications. Potential applications include, but are not limited to, treating chronic wounds, surgical interventions, and regenerating damaged tissues and organs.

In some implementations, a single-use, disposable solution ampule may be used with the device to apply a non-toxic, biocompatible polymer and highly efficacious antimicrobial additives to create a complex force-spun, water-soluble matrix that mimics natural soft tissue physiology on the nano- and cellular scale. The device may preferably apply a coating of a nanofiber matrix on the affected area which is transparent, gentle, and breathable. The nanofiber matrix may preferably incorporate antimicrobial agents. The nanofiber matrix may preferably be applied without contacting the burned tissue. Contactless application of the nanofiber matrix may preferably result in a pain-free or minimal pain treatment. Notably, application of the nanofiber matrix is gauze-free and adhesive-free, and does not require surgery. The application of the nanofiber matrix onto burned tissue may result in improved tissue regeneration and may overcome the painful steps of skin grafting and traditional wound dressing.

The disclosed device may be used to spray a nanofiber healing matrix onto wounds, mimicking natural skin and accelerating the healing process.

The device is preferably portable and may preferably be used in uncontrolled environments. This provides significant utility in emergency situations.

In some implementations, the nanofiber matrix may preferably incorporate one or more antibacterial additives. In some implementations, the one or more antibacterial additives include licorice extract.

The incorporation of antibacterial additives reduces the risk of infection without painful re-dressing or contact.

The nanofiber matrix is preferably transparent and lightweight, allowing for easy monitoring of the healing process.

The disclosed device may be used to apply a nanofiber matrix to the entire body in a single application. In some implementations, the device may be used to treat up to 1.9 m² of surface area in a single application, which corresponds to the entire body surface of an average adult human.

Use of the disclosed device to apply a nanofiber matrix allows for proper adherence even for hard-to-dress contours. Further, use of the disclosed device to apply a nanofiber matrix enables application of a wound dressing and healing coating that is specifically tailored to the needs of the burn patient with respect to wound size, wound geometry, degree of burning, and other patient-specific parameters without requiring large stocks of dressings of various shapes and sizes.

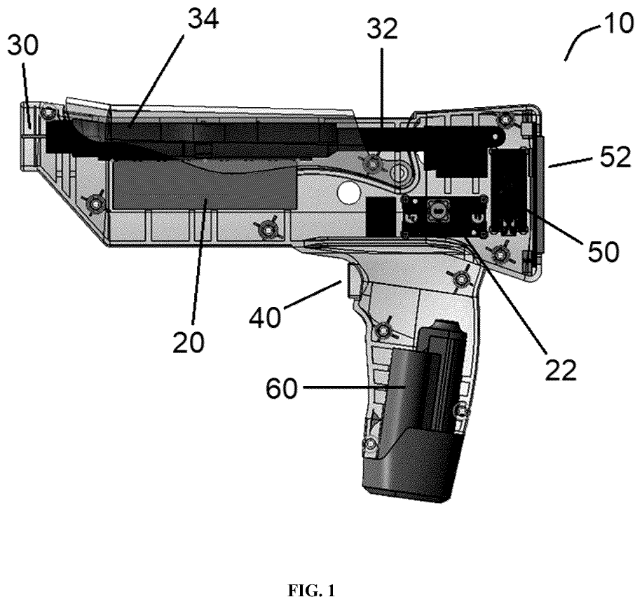

FIG. 1 shows an embodiment of the disclosed device that uses an electrospinning apparatus incorporated into a handheld application gun 10. The application gun 10 includes a high voltage power supply 20 having positive and negative electrodes (not labeled), where the positive electrode is operationally connected to a needle 30 via a housing that surrounds the needle (not labeled) and the negative electrode is connected to an external port (not labeled) to which an external ground may be attached. The application gun 10 includes a start button 40 which is used to start and stop the electrospinning process. The application gun 10 includes a voltage regulator 22 that regulates the voltage generated by the high voltage power supply 20. A controller 50 is operationally connected to a display 52 that allows a user to monitor the voltage and other parameters. A battery 60 provides the necessary power for components that require power. A linear actuator 32 controls release of a solution for electrospinning from a syringe (not shown) that is housed in a syringe holder 34 into the needle 30. The start button 40 is operationally connected to the linear actuator 32.

The handheld gun design allows for intuitive usage, making the device suitable for various biomedical applications. The ergonomic grip of the handle ensures comfortable operation, while the controls and indicators may be strategically placed for effortless monitoring and adjustment.

In some implementations, the device uses an electrospinning apparatus that includes a high voltage power supply that generates voltages up to about 25 kV. This allows users to perform electrospinning from a reasonable distance from the application site of the electrospun nanofiber matrix. A user can create a nanofiber matrix without getting too close to the actual burnt tissue, which expands the range of possible applications.

In some other implementations, the device uses an electrospinning apparatus that includes a high voltage power supply that generates voltages up to about 10 kV. This allows the device to incorporate a smaller power supply and increases portability.

In some implementations, the electrical voltage output is proportional to the force applied when pressing the start button on the device. This enables a user to adjust the electrospinning intensity based on the pressure applied to the start button. This increases control over and precision of the electrospinning process.

In some implementations, the device includes an external port to which an external ground may be attached. The external ground may be linked to the patient to enhance safety by preventing unintended charge transfer to the patient during the nanofiber deposition process.

In some implementations, the device uses alternating current (AC) instead of direct current (DC) for electrospinning. This eliminates the need to link the patient to an external ground, which simplifies device setup and use, and which also improves safety and patient comfort.

In some implementations, the device may be switched between operating using AC and DC.

In some alternate implementations, a corona ion neutralizer is used to neutralize charge on the deposited fiber, thereby reducing net charge accumulation at the deposition site. In some other alternate implementations, a self-balancing bipolar electrode delivers charge of substantially balanced polarity, thereby reducing or preventing net charge accumulation on the deposited fiber. In both such alternate implementations, the device is configured to reduce net charge accumulation by neutralizing accumulated charge, delivering charge of balanced polarity, or both. These alternate implementations may reduce or eliminate the need for an external ground.

In some implementations, the device uses an electrospinning apparatus that includes a ring that may be positioned near the front of the needle or other nozzle used for electrospinning. The ring has the same positive charge as the needle or other nozzle. This ring narrows and focuses the electrospinning jet, facilitating more accurate targeting of the patient's wound. By adjusting the distance between the ring and the tip of the nozzle, it is possible to adjust the width of the electrospinning jet. This adjustability allows a user to balance between focus and coverage, optimizing the balance for the specific application.

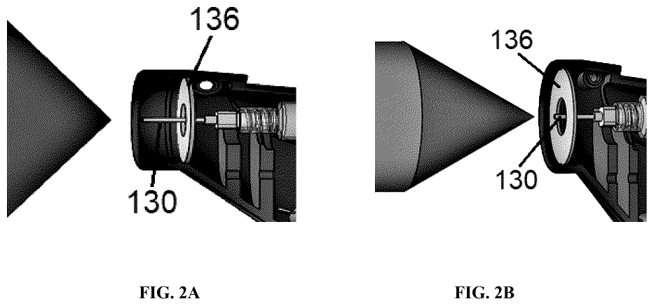

FIGS. 2A and 2B show embodiments of a ring 136 positioned at variable distances from the front of the needle 130 used for electrospinning.

In some implementations, a blower or heater is positioned exterior to the needle or other nozzle to enable pre-drying of fibers generated before the fibers contact the target. Pre-drying the fibers may cause the electrospun matrix to be more uniform and smooth by eliminating defects caused by residual moisture.

In some implementations, an ultrasonic or laser sensor is used to measure the spinning distance to allow for dynamic adjustment of the applied voltage. By ensuring the spinning distance remains within the optimal range and automatically shutting off the electric field when deviations beyond the optimal range occur, greater consistency in fiber production may be achieved.

In some implementations, a light source is used to visually identify the optimal spinning distance. In some implementations, the light source may cast a spot having a first color and first shape on the target tissue to indicate that the spinning distance is within the optimal range of distances, may cast a spot having a second color or second shape when the spinning distance is less than the optimal range of distances, and may cast a spot having a third color or third shape when the spinning distance is greater than the optimal range of distances. In some alternate implementations, the light source may cast two spots on the target tissue, where the two spots converge into a single spot when the spinning distance is within the optimal range of distances. The light source may, for example, be a laser.

In some implementations, ultrasonic waves are applied to the needle or other nozzle to facilitate overcoming the surface tension of the solution, leading to a more efficient and productive electrospinning process. Application of ultrasonic waves will not only increase output but also enhance the overall quality of the fibers produced.

In some implementations, the handheld device includes a disposable cone that mechanically sets the correct distance for electrospinning the nanofiber matrix.

In some alternate implementations, the device includes an electro-centrifugal spinning apparatus. The electro-centrifugal spinning apparatus is integrated into an application gun that is analogous to the application gun described above. The electro-centrifugal spinning apparatus may include a spinneret that is operationally coupled to multiple nozzles for electro-centrifugal spinning. In some implementations, the electro-centrifugal spinning apparatus may be configured to generate an electric field of up to 30 kV to charge the electro-centrifugally spun fibers and direct them toward a desired target. The electro-centrifugal spinning apparatus may further include a rotation arm that causes rotation of the nozzles and controlled ejection of the fibers. A power supply may be included to facilitate the application of an electric field during rotation to enhance fiber adhesion to targeted tissues or another desired target. The electro-centrifugal spinning apparatus may also include a user interface for adjusting rotation parameters, including electric field intensity and rotation speed.

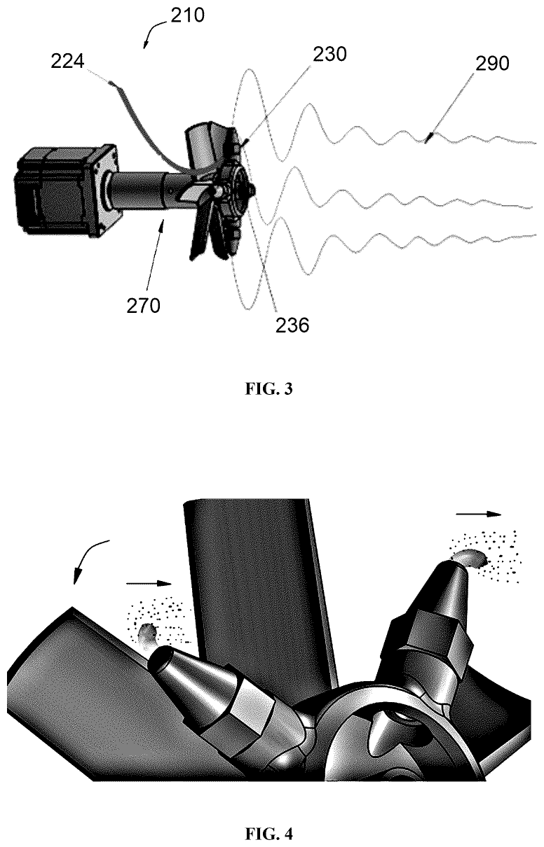

FIG. 3 shows an embodiment of the disclosed electro-centrifugal spinning apparatus 210. A high voltage input connector 224 is connected to a power supply (not shown) for generating a high voltage during rotation. Multiple nozzles 230 are connected to a spinneret 236. A rotation arm 270 causes the nozzles to rotate around the spinneret when the apparatus is in use. Use of the electro-centrifugal spinning apparatus generates multiple jets 290 for placement or adhesion onto a desired target.

In some implementations, the rotation arm may be controlled to adjust the spin rate across the multiple nozzles. The adjustable rotation speed precisely guides a polymer solution into the openings through centrifugal force. In addition, the fibers are charged via application of high voltage, facilitated by the rotated droplet shape of the polymer solution as it emerges from the nozzle. This allows breaking of the surface tension of the solution, significantly amplifying both the capacity and production efficiency of nanofibers.

FIG. 4 shows a close-up sectional view of the embodiment shown in FIG. 3, illustrating how a polymer solution being spun will be affected by rotation of the rotation arm when the polymer solution exits a nozzle.

By combining the precise control provided by electrospinning with the increased throughput provided by centrifugal spinning, the electro-centrifugal spinning apparatus allows for precise, efficient delivery of a nanofiber matrix to a desired target.

The nozzles are guiding modules for desired polymer matrices to coat the target surface and construct layered matrices. In implementations with multiple nozzles, the disclosed application gun has at least two integrated chambers. At least one chamber may be used for spinning a polymer solution and at least one chamber may be used for applying a suspension. The disclosed application gun may be used to apply both the suspension and the polymer solution simultaneously to construct layered matrices of suspension/polymer on the applied surfaces or apply the suspension and polymer solutions separately to provide an initial layer of polymer and a subsequent layer of the suspension. Both processes may be used repeatedly to achieve multi-layered structures to build a desired thickness. This offers flexibility in constructing a desired matrix using different polymers and suspensions.

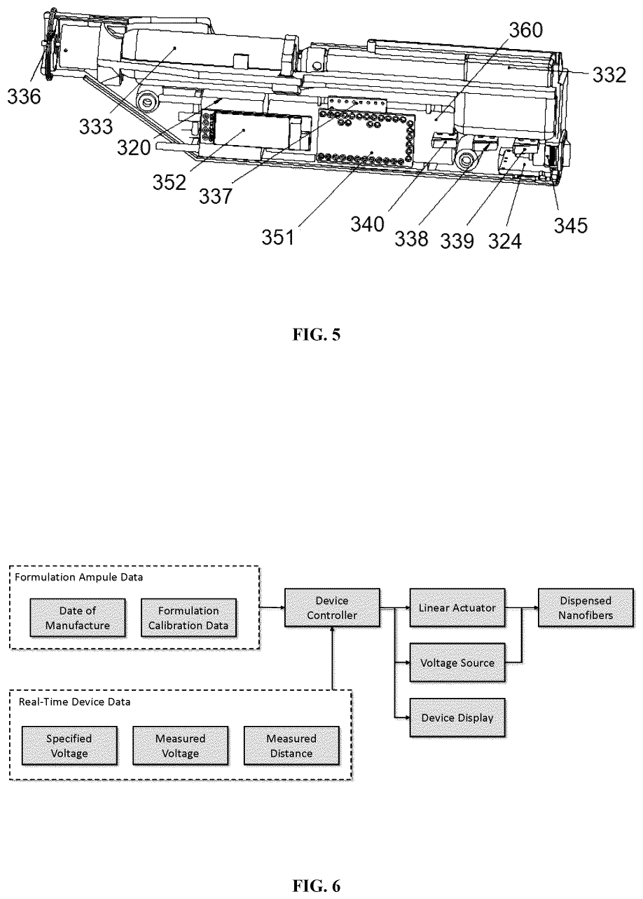

FIG. 5 shows an embodiment of the disclosed device that uses an electrospinning apparatus incorporated into a handheld applicator 310. The applicator 310 includes a high voltage power supply 320 having positive and negative electrodes (not labeled), where the positive electrode is operationally connected to a needle (not labeled) via a housing that surrounds the needle (not labeled) and the negative electrode is connected to an external port (not labeled) to which an external ground may be attached. The applicator 310 includes a switch 340 which is used to start and stop the electrospinning process by turning the power supply 320 on or off. The applicator 310 includes a voltage regulator (not labeled) that regulates the voltage generated by the high voltage power supply 320. A battery 360 provides the necessary power for components that require power. A battery charger and external power supply 324 is used to charge the battery and provide power to the circuit. A microcontroller 351 is operationally connected to a display 352 that allows a user to monitor the voltage, flow rate, battery status, and other parameters, and is also operationally connected to other components of the device to allow the user to control operating parameters. A linear actuator 332 controls release of a solution for electrospinning from a syringe 333 that is housed in a syringe holder (not labeled) into the needle. A directional ring 336 positioned near the front of the needle that has the same positive charge as the needle allows narrowing and focusing of the electrospinning jet. A step motor driver 337 provides mechanical control to actuate linear actuator 332. Microswitches 338 and 339 are operationally connected to the step motor driver 337 to provide for left and right adjustments thereof, respectively. A system switch 345 is used to turn the device on or off.

In some implementations, the device includes a microprocessor that is used to adjust operating parameters algorithmically. FIG. 6 shows an example of a set of parameters that are communicatively coupled to a microcontroller, where operating parameters are adjusted algorithmically. In some implementations, only a subset of the data inputs shown in FIG. 6 are used. In an illustrative implementation, the operating voltage and linear actuator are modulated by the measured voltage, the age of the polymer solution in the formulation ampule, and formulation calibration data through an algorithm that enables optimal performance. In some implementations, the polymer solution data is encoded via a bar code or QR code, while in other implementations the ampule is communicatively coupled to the microcontroller by, for example, an EEPROM or other non-volatile memory source.

In some implementations, the device includes a laser-enabled nanofiber thickness detection system. The thickness detection system enhances the precision and consistency of the nanofiber deposition process. A laser interacts with the nanofiber layer and measures the reflection, refraction, and interference patterns, enabling the system to accurately determine the thickness of the layer in real time. This not only ensures optimal nanofiber deposition but also allows for immediate adjustments, optimizing both quality and efficiency in application of the wound dressing.

In some implementations, the device includes an onboard camera that images a wound, where the onboard camera is operationally connected to a controller that receives images from the camera and computes a surface area of the wound to determine the spinning time and number of iterations required to deposit a user-selected matrix thickness (areal density). In some implementations, the controller causes the device to automatically stop spinning upon achieving the user-selected matrix thickness. In some implementations, a fiducial may be used as a reference to determine how many iterations are required to deposit a matrix of the selected thickness. In some implementations, the controller may recompute the spinning time and number of iterations required multiple times during the deposition of the matrix. In some implementations, the camera may be a structured light camera or a stereo camera.

In some implementations, the onboard camera further tracks the deposited matrix in real time and generates images showing regions of the targeted wound where a nanofiber matrix has been deposited and the thickness of the deposited matrix in those regions. This allows a user of the handheld device to identify gaps to ensure more uniform deposition of the nanofiber matrix.

In some implementations, the nozzle or electrospinning jet may be controlled by a gimbal or a beam steering component to follow the contours of irregular or deep wounds for even coverage on non-flat wound surfaces.

In some implementations, co-located ultraviolet or blue light may be used during electrospinning for in situ crosslinking or to provide an antimicrobial effect.

In some implementations, a simultaneous antiseptic or anesthetic micro-spray is applied to the nanofiber matrix through a second channel during electrospinning.

In some implementations, the handheld device includes an inertial measurement unit (IMU) to detect excessive device movement. The IMU pauses or modulates the field, or alternately maintains a steady sweep, as needed.

In some implementations, the handheld device includes an onboard temperature or humidity sensor.

In some implementations, the handheld device includes a controller that is configured to auto-adjust the voltage, flow rate, and/or standoff during electrospinning to maintain consistent fiber morphology across varying ambient conditions.

In some implementations, a single-use, pre-filled sterile solution cartridge carrying an RFID, barcode, or key is provided. The handheld device is configured to read the RFID, barcode, or key and auto-load validated parameter presets—such as voltage, flow rate, distance, and time—for that exact formulation.

In some implementations, a controller records electrospinning parameters, computed area, delivered dose, and images pre- and post-electrospinning. The controller is configured to transmit this data to an electronic health record (EHR) system to support remote supervision, telemedicine, or regulatory documentation.

A method of controlling the thickness of a nanofiber matrix applied to a wound using the disclosed handheld device is also disclosed herein. The method may include the steps of imaging the wound using a camera, transmitting the images to a controller, determining using the controller an area and spinning time required to apply a nanofiber matrix of a selected thickness to the wound, and depositing the nanofiber matrix using the handheld device. In some implementations, the method may further include the step of automatically stopping the further deposition of nanofiber matrix layers upon achieving the selected thickness. In some implementations, the method further includes the steps of generating real-time images of the nanofiber matrix during deposition, transmitting the real-time images to the controller, identifying using the controller one or more regions which require further deposition of nanofiber matrix layers, and depositing additional nanofiber matrix layers in the one or more identified regions. In some implementations, the method includes use of a gimbal or a beam steering component to follow the contours of irregular or deep wounds to generate a nanofiber matrix that evenly covers the wound surface.

In some alternate implementations, the thickness of the deposited matrix may be measured in real-time using an optical, capacitive, or impedance sensor. In some implementations, a controller causes the device to automatically stop spinning upon achieving a user-selected matrix thickness that is determined using an optical, capacitive, or impedance sensor.

An alternate method of controlling the thickness of a nanofiber matrix applied to a wound using the disclosed handheld device is also disclosed herein. The method may include the steps of determining the thickness of the matrix using an optical, capacitive, or impedance sensor; transmitting the determined thickness to a controller; periodically repeating the steps of determining the thickness of the matrix and transmitting the determined thickness to a controller until a selected thickness is achieved; and automatically stopping the further deposition of nanofiber matrix layers upon achieving a selected thickness.

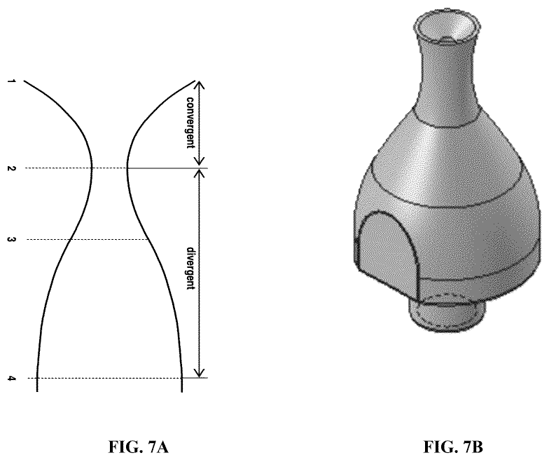

In some implementations, the device includes a convergent-divergent nozzle. FIG. 7A shows a schematic representation of the configuration of a convergent-divergent nozzle. FIG. 7B shows an example of a convergent-divergent nozzle. The convergent-divergent nozzle is shaped to promote a polymer solution therein to adopt a cone shape.

In some implementations, the device is capable of being operated by user when the device is held by the user with one hand.

The polymer solution used for electrospinning or electro-centrifugal spinning may be a water or ethanol-based formulation that includes one or more of the polymer materials selected from the group consisting of polyvinyl alcohol (PVA), polyvinyl butyral (PVB), chitosan, collagen, hyaluronic acid (HA), B-silk, or a blend of any combination thereof. The formulation may further include an herbal extract that has antimicrobial properties, preferably licorice extract. The formulation may alternately or additionally include other biologics. The formulation may also further include antibiotics, painkillers, and/or other drugs.

By blending antimicrobials, antibiotics, painkillers, and/or other drugs or other biologics with the polymer solution, the nanofiber matrix generated may directly incorporate biologics for enhanced wound healing.

Biologics included in the polymer solution may include autologous or allogenic cell suspensions, including keratinocytes for epidermis regeneration, dermal fibroblasts for new extracellular matrix protein deposition, and/or melanocytes for natural pigmentation restoration. Biologics may additionally or alternately include platelet-rich plasma (PRP).

In some implementations, biologics may be blended with the polymer formulation in a single step, thereby allowing the biologics to be seamlessly incorporated into the nanofibers as they are spun. This method of blending will ensure a more uniform and effective distribution of the biologics within the nanofiber matrix, potentially improving the healing properties thereof. This single-step integration may streamline the process of including biologics in the nanofiber matrix, making it more efficient and potentially more effective in wound healing applications.

In some implementations, biologics may alternately be blended via sequential layering, where the disclosed device will sequentially apply multiple layers of material incorporating biologics and nanofibers. This may be achieved by having an additional nozzle for spraying biological substances (cells or PRP), followed by nanofiber spinning, and repeating the process as necessary.

In some implementations, biologics may alternately be applied using a different device, such as a syringe, if integration with the disclosed spinning device is not feasible.

In some implementations, the polymer solution may be used to deliver a non-toxic, non-reactive, and sustainable cosmetic PVA-based nanofiber matrix. The dissolution of PVA depends on solvent, temperature, and the extent of hydrolysis within the polymer. Additionally, solution parameters such as solution concentration, molecular weight, pH, salt, and surfactant molecules affect the morphology and diameter of electrospun PVA nanofibers. Thus, a series of PVA polymers (5-10% wt %) with selected molecular weight and degree of hydrolysis (60-99.9%), along with surfactants such as polyvinylpyrrolidone (PVP) and polysorbate, may be dissolved in a series of solvents such as water and water/ethanol mixtures (1/1, 2/1, and 3/1) to generate a PVA-based nanofiber matrix for cosmetic applications. Various active ingredients such as collagen peptides, vitamin A, and vitamin C, may also be blended into the polymer solution prior to spinning.

### EXAMPLE

Licorice Extract Preparation. Licorice powder was mixed with a solvent consisting of ethanol and water in a 1:1 ratio, with a weight ratio of 1:3 powder/solvent. The mixture was placed in a sealed container and stirred continuously for 5 hours at ambient temperature, ensuring regular agitation to maximize the extraction of active compounds such as glycyrrhizin and flavonoids. Post-extraction, the mixture was filtered to separate the liquid extract from the solid residues, yielding a crude licorice extract. A rotary evaporator was used under reduced pressure to concentrate the liquid extract, resulting in a thick, concentrated extract.

Formulation/Solution Preparation. Polyvinyl alcohol (PVA) was dissolved in a water/ethanol mixture at a concentration of 9% wt/v with an 80:20 ratio at ambient temperature. To this solution, licorice extract prepared as described above was added at 0.1% wt/v, followed by the addition of phosphate-buffered saline (PBS) at 0.05% wt./v. The entire mixture was then stirred on a magnetic stir plate for two hours to ensure homogeneity.

Electrospinning using Handheld Applicator. The prepared solution was loaded into a 5 mL syringe, which was then inserted into a handheld applicator. The applicator included a ring positioned to allow the syringe needle to be inserted through the ring, and 5 mm of the needle to extend beyond the ring. Electrospinning was performed at a flow rate of 500 μL/min, an applied voltage of 10 kV, and a distance of 10 cm between the applicator and the target surface.

In a typical electrospinning setup, a high voltage (typically above 15 kV is required to overcome the surface tension of the polymer solution and initiate spinning. Achieving such high voltage in a compact, handheld device is challenging, as a larger power supply must be used to generate higher voltages. A 10 kV power supply is generally insufficient for electrospinning, particularly with water-based solutions due to their low conductivity and low volatility, as these are critical parameters for successful electrospinning.

The example above shows successful electrospinning at a voltage of 10 kV. This was possible due to synergistic effects of the electrospinning parameters used, namely increased volatility of the solution generated by the addition of ethanol, increased conductivity generated by the use of PBS, and focusing of the electric field by use of a positively-charged ring. This approach allows for effective electrospinning in a handheld device at a reduced voltage, solving the key challenge of miniaturizing the power supply required while maintaining functionality.

Other experiments were carried out under similar conditions, without the use of PBS or ethanol and using a higher voltage of 25 kV during electrospinning. These handheld electrospinning devices required a larger power supply and therefore the devices were larger overall.

### NUMBERED EXAMPLES

### Example 1

A method of applying a nanofiber matrix to a wound composed of electrospinning a polymer solution using a handheld electrospinning device to generate a nanofiber matrix,
where the polymer solution includes:
   a. polyvinyl alcohol;
   b. licorice extract;
   c. phosphate-buffered saline;
   d. water; and
   e. ethanol; and
where electrospinning is performed using an applied voltage of about 10 kV.

### Example 2

A device for application of an electrospun nanofiber matrix to a wound that includes:
a. a power supply;
b. a syringe that includes a needle; and
c. a microcontroller;
   where the power supply is operationally connected to the needle,
   where the microcontroller is configured to allow real-time adjustment of electrospinning parameters, and
   where the device may be operated by a user using one hand to hold the device.

### Example 3

The device of Example 2 further including a ring positioned around or in front of the needle.

### Example 4

The device of Example 3, where the ring is positioned around the needle.

### Example 5

The device of Example 3, where the ring is positioned in front of the needle.

### Example 6

The device of any of Examples 3-5, where the ring is composed of a metal.

### Example 7

A device for application of an electrospun nanofiber matrix to a wound that includes:
a. a power supply;
b. a syringe that includes a needle;
c. a ring positioned around or in front of the needle; and
d. a microcontroller;
   where the power supply is operationally connected to the needle, and
   where the device may be operated by a user using one hand to hold the device.

### Example 8

The device of Example 7, where the ring is positioned around the needle.

### Example 9

The device of Example 7, where the ring is positioned in front of the needle.

### Example 10

A device for application of an electrospun nanofiber matrix to a wound that includes:
a. a power supply;
b. an electro-centrifugal spinning apparatus; and
c. a microcontroller;
   where the power supply is operationally connected to the electro-centrifugal spinning apparatus, and
   where the device may be operated by a user using one hand to hold the device.

### Example 11

The device of Example 10, where the electro-centrifugal spinning apparatus includes a spinneret that is operationally coupled to multiple nozzles for electro-centrifugal spinning.

### Example 12

The device of Example 11, where the electro-centrifugal spinning apparatus further includes a rotation arm that enables rotation of the nozzles.

### Example 13

The device of any of Examples 10-12, where the microcontroller is configured to allow real-time adjustment of electrospinning parameters.

The previous description of the disclosed embodiments is provided to enable any person skilled in the art to make or use the invention disclosed herein. Although the various inventive aspects are disclosed in the context of certain illustrated embodiments, implementations, and examples, it should be understood by those skilled in the art that the invention extends beyond the specifically disclosed embodiments to other alternative embodiments and/or uses of the invention and obvious modifications and equivalents thereof. In addition, while a number of variations of various inventive aspects have been shown and described in detail, other modifications that are within their scope will be readily apparent to those skilled in the art based upon reviewing this disclosure. It should be also understood that the scope of this disclosure includes the various combinations or sub-combinations of the specific features and aspects of the embodiments disclosed herein, such that the various features, modes of implementation, and aspects of the disclosed subject matter may be combined with or substituted for one another. The generic principles defined herein may be applied to other embodiments without departing from the spirit or scope of the disclosure. Thus, the present disclosure is not intended to be limited to the embodiments shown herein but is to be accorded the widest scope consistent with the principles and novel features disclosed herein.

Each of the foregoing and various aspects, together with those summarized above or otherwise disclosed herein, including the figures, may be combined without limitation to form claims for a device, apparatus, system, method of manufacture, and/or method of use.

All references cited herein are hereby expressly incorporated by reference.

What is claimed is:

1. A method of applying a nanofiber matrix to a wound comprising electrospinning a polymer solution using a handheld electrospinning device to generate a nanofiber matrix,
wherein the polymer solution comprises:
a. polyvinyl alcohol;
b. licorice extract;
c. phosphate-buffered saline;
d. water; and
e. ethanol; and
wherein electrospinning is performed using an applied voltage of about 10 kV.

2. A device for application of an electrospun nanofiber matrix to a wound comprising:
a. a power supply;
b. a syringe that includes a needle; and
c. a microcontroller;
wherein the power supply is operationally connected to the needle,
wherein the microcontroller is configured to allow real-time adjustment of electrospinning parameters, and
wherein the device may be operated by a user using one hand to hold the device.

3. The device of claim 2 further comprising a ring positioned around or in front of the needle.

4. The device of claim 3, wherein the ring is positioned around the needle.

5. The device of claim 3, wherein the ring is positioned in front of the needle.

6. The device of claim 3, wherein the ring is composed of a metal.

7. The device of claim 4, wherein the ring is composed of a metal.

8. The device of claim 5, wherein the ring is composed of a metal.

9. A device for application of an electrospun nanofiber matrix to a wound comprising:
a. a power supply;
b. a syringe that includes a needle;
c. a ring positioned around or in front of the needle; and
d. a microcontroller;
wherein the power supply is operationally connected to the needle, and
wherein the device may be operated by a user using one hand to hold the device.

10. The device of claim 9, wherein the ring is positioned around the needle.

11. The device of claim 9, wherein the ring is positioned in front of the needle.

12. A device for application of an electrospun nanofiber matrix to a wound comprising:
a. a power supply;
b. an electro-centrifugal spinning apparatus; and
c. a microcontroller;
wherein the power supply is operationally connected to the electro-centrifugal spinning apparatus, and
wherein the device may be operated by a user using one hand to hold the device.

13. The device of claim 12, wherein the electro-centrifugal spinning apparatus comprises a spinneret that is operationally coupled to multiple nozzles for electro-centrifugal spinning.

14. The device of claim 13, wherein the electro-centrifugal spinning apparatus further comprises a rotation arm that enables rotation of the nozzles.

15. The device of claim 12, wherein the microcontroller is configured to allow real-time adjustment of electrospinning parameters.

16. The device of claim 13, wherein the microcontroller is configured to allow real-time adjustment of electrospinning parameters.

17. The device of claim 14, wherein the microcontroller is configured to allow real-time adjustment of electrospinning parameters.
